(19) United States
(12) Patent Application Publication
(10) Pub. No.: US 2025/0012729 A1
(43) Pub. Date: Jan. 9, 2025

(54) MILKING SYSTEM WITH SAMPLING AND ANALYSIS

(71) Applicant: LELY PATENT N.V., Maassluis (NL)

(72) Inventors: Johannes Adriaan DRONKERT, Poortugaal (NL); Leendert DE BORST, Abbenes (NL); Frans Jacob DE GRAAF, Nieuw Vennep (NL)

(73) Assignee: LELY PATENT N.V., Maassluis (NL)

(21) Appl. No.: 18/710,720

(22) PCT Filed: Dec. 8, 2022
(86) PCT No.: PCT/IB2022/061931

§ 371 Date: May 16, 2024

(30) Foreign Application Priority Data
Dec. 13, 2021 (NL) 2030112

(51) Int. Cl. G01N 21/78; A01J 5/007; G01N 33/14

(52) U.S. Cl. CPC G01N 21/78; A01J 5/007; G01N 33/14; G01N 2201/062; G01N 2201/0636

(57) ABSTRACT

A milking system includes a milking device, a sampling device and an analyser for analysing a milk sample, with a housing for a first holder with a reagent carrier with indicator pads, and a second holder for used reagent carrier, a metering system, a first optical radiation source for optical imaging radiation, a sensor device for detecting and analysing response radiation derived from the active indicator pad, for supplying an indication of a presence or concentration of said at least one substance in the sample. The analyser further includes a temperature control system for keeping the active indicator pad at temperature, with a second optical radiation source for emitting optical heating radiation, and a source control device to control the second optical radiation source.

The present invention relates to a milking system, comprising a milking device with milking means and a milking control device, and configured to extract milk from a milking animal, a sampling device that is configured to take a sample of the milk extracted by the milking device, and an analyser that is configured to analyse the sample, wherein the analyser comprises a housing for receiving at least one first holder with a reagent carrier with indicator pads applied thereon, as well as for receiving a second holder for collecting used reagent carrier, wherein the indicator pads comprise a reagent that in the presence of at least one substance in the milk of the sample gives a detectable reaction, and wherein the first holder and the second holder are preferably assembled in an interchangeable cassette, a reaction space provided within the housing between the first holder and the second holder, for containing a part of the reagent carrier with at least one active indicator pad, a metering system for supplying the sample taken to one of the cassettes, a first optical radiation source for emitting optical imaging radiation to said active indicator pad, and an optical sensor device configured to detect optical response radiation that comes from the active indicator pad in response to the emitted optical imaging radiation, and to analyse the detected optical response radiation for supplying an indication of a presence or concentration of said at least one substance in the sample.

A milking system of this kind is known for example from WO2020/067882 of the applicant. Milking systems of this kind are able to sample and analyse the milk from a milking animal, and based thereon, improve the milking or in general the management of the animal. Thus, a deviation in the milk can become evident earlier from such an analysis than from the analysis of milk samples sent to a laboratory at regular points in time. The farmer can then take corrective measures more quickly. It is also possible that the analysis is quick enough to adjust the destination of the milked milk, or at least that of the milk from subsequent milking operations. It is of course important that the analysis takes place quickly and reliably.

A drawback of the known device is that the reaction of some reagents is relatively temperature-dependent, so that without further control, the accuracy of the analysis may leave something to be desired. This is of course undesirable when this analysis is used for the management of milking animals. However, milking systems are often accommodated in milking parlours that are in communication with the outside air. As a result, milking systems are exposed to much larger temperature variations than for example laboratory equipment, such as above 30° C. on a warm summer's day, even to negative temperatures in winter.

Now it is known per se to heat the whole analyser, or at least the housing, to the optimal temperature. However, there are also disadvantages associated with this, such as the large amount of energy required for heating the housing, but also for example the low flexibility of the system. Moreover, a different reagent may have an optimal reaction at a different temperature. In addition, heating and cooling of a whole housing takes longer, and especially if a plurality of cassettes are placed in the housing, a compromise would have to be found perhaps unnecessarily.

The aim of the invention is to provide a milking system of the kind stated in the introduction that does not have said drawbacks, or at least has them to a lesser extent.

For this purpose, the invention provides a milking system as claimed in claim 1. The analyser further comprises a temperature control system that is configured to bring or maintain the active indicator pad at a desired temperature, and that comprises a second optical radiation source that is configured to emit optical heating radiation at a first solid angle, a concentrator for concentrating the emitted optical heating radiation at a smaller second solid angle and onto the active indicator pad, and a source control device that is configured to control the second optical radiation source.

The milking system according to the invention is able, by means of optical heat radiation, to provide directed heating of an indicator pad. As a result, the total mass to be heated, and therefore the amount of energy required, are minimal. Moreover, optical heat radiation is easily controllable, so that there is far less warming of the surroundings of the irradiated indicator pad, and therefore no adverse consequences. Optical heating of this kind thus has advantages relative to heating with for example heated air, because it will also have to flow along the indicator pad. There, however, the air will also affect the air humidity, which is also undesirable, to avoid affecting the reaction kinetics. Heating by conduction has in its turn the drawback that it takes place indirectly, and is therefore much slower, and by heating a relatively large mass. In addition, during the reaction with the milk sample, the indicator pad is not itself in contact with any other object, so that heating of said indicator pad by conduction is not in fact even possible. The choice of the temperature control system is thus associated with quite a lot of boundary effects.

It is noted here that, just as with the known device, the metering system used in the present invention may partly be present in a cassette. That is, for example a dropper with a feed tube and pump is provided in the cassette, and thus interchangeable, wherein the cassette is connected to a sample feed tube from the sampler/analyser. It is of course equally possible that the whole metering system is provided as a fixed component in the milking system, so that the cassette can be regarded as purely a reagent carrier. Either is suitable for application of the invention.

The optical response radiation may be reflected, transmitted, fluorescent or diffuse, but that too makes no difference for application of the invention.

Particular embodiments of the invention are described in the dependent claims, as well as in the part of the description that now follows.

In some embodiments, the first optical radiation source and the second optical radiation source are provided within the housing, outside any cassette, and each cassette comprises a window which, when using the milking system, allows the optical imaging radiation and/or the optical heating radiation to pass through. A cassette comprises a tape with indicator pads, as well as a window to allow the optical radiation to pass through. This radiation refers in these embodiments both to the imaging radiation, i.e. the radiation with which the reaction in the indicator pad can be examined, and which leads to the optical response radiation that is received and analysed by the optical sensor device, and to the optical heating radiation. Of course, the response radiation should be allowed through, but in by far the most cases this will comprise at least a part of the frequencies of the optical imaging radiation. It is only in the case of fluorescence radiation that these frequencies may deviate, and become lower. In such cases it is to be noted that the window should also allow the desired fluorescent radiation to pass through. By providing both radiation sources outside the cassette, these only need to be provided once within the housing, so that the cassettes can be changed freely.

The optical imaging radiation may in principle be selected freely from the available optical radiation, i.e. electromagnetic radiation with a wavelength between 100 nm and 1 mm. In particular, this optical radiation will, however, be selected from visible radiation and optionally near-infrared radiation, with a wavelength between about 380 and 1000 nm. The optical sensor device is then advantageously, but not exclusively, a video camera.

The optical heat radiation may also in principle be selected from the available optical radiation, with the same wavelength range. However, it is desirable that the selected radiation can heat the material of the reagent carrier, thus the tape and the indicator pad, effectively enough. That is possible with visible radiation, such as red light, but it seems advantageous to do this with near-infrared light, since many materials display a high, or even optimal absorption, in that wavelength range.

In some embodiments, the temperature control system is configured to have at any time at most one of the first optical radiation source and the second optical radiation source in operation. This in principle excludes influence on the optical sensor device from the optical heating radiation, and possibly also vice versa. However, it is also possible to have both optical radiation sources in operation simultaneously, provided that undesirable effects are counteracted by means of filtering or the like. Thus, a possible, but often occurring, sensitivity of the optical sensor device in the near-infrared can be counteracted by fitting a (near-)infrared filter.

In some embodiments the temperature control system comprises a thermometer configured for determining a temperature of the active indicator pad, and is configured to control the second optical radiation source on the basis of the temperature determined. The thermometer measures or determines/estimates the temperature of the indicator pad for example by contact or preferably contactlessly, such as a radiation thermometer. The temperature value obtained is fed to the source control device, which determines whether the desired temperature has already been reached. If not, it switches on the second optical radiation source, or increases the power emitted and/or extends the time that the second optical radiation source supplies its radiation, until the measured temperature matches the desired temperature. This temperature is, as mentioned, dependent on the reagent in the indicator pad. In many cases the desired temperature is roughly 37° C., but may also for example be at “standard room temperature” of between 2° and 25° C.

In alternative or supplementary embodiments the temperature control system is configured to emit the optical heating radiation for a predetermined length of time, preferably depending on the reagent. The heating may be regulated with the thermometer, but if the properties of the indicator pads and those of the second optical radiation source are well known, it is also possible to predict the temperature on the basis of the heating time. Thus, no thermometer is necessary, just a clock, which is already present in the control device.

In some embodiments, the temperature control system further comprises a second thermometer for determining an ambient temperature, and the temperature control system is configured to control the second optical radiation source on the basis of the ambient temperature determined. Especially for the heating system based on duration, the influence of the ambient temperature is sometimes considerable. That will determine the starting temperature of the indicator pad, and at a high ambient temperature the desired temperature will be reached much earlier.

The second optical radiation source is in principle not particularly limited. To be able to direct the radiation well, it is desirable that the dimensions of the source are limited. Thus, for example fluorescent lighting or other diffuse radiation will be unsuitable. For example, it is possible to use a halogen lamp. In particular, however, the second optical radiation source comprises an LED, in particular a near-infrared LED. Not only are the dimensions of an LED of this kind even smaller than those of a halogen lamp, so that the radiation can be directed even better, but in addition the efficiency of an LED is higher than that of a halogen lamp, and an LED is in addition much quicker to switch on. An LED is off almost immediately, but a halogen lamp always displays some glow after switch-off, and the effects on the optical sensor device may have undesirable consequences. Counteracting this at least requires either filtering, or a sufficient waiting time, both of which are unnecessary when using an LED. The LED is advantageously, but not exclusively, a near-infrared LED, which with narrow-band near-infrared light reaches a relatively high efficiency.

Advantageously, the concentrator is a concave mirror or a lens. These are useful means, well known per se, for directing and concentrating optical radiation. Even in the near-infrared region, sufficient materials are known for making effective lenses and mirrors.

In some embodiments, said second solid angle has a widest apex angle of at most 15°, preferably at most 10°. It will be clear that a narrower solid angle generally leads to both more intense optical heating radiation and a more directional beam, so that there is less effect on the surroundings. This allows e.g. a more compact construction, which is especially advantageous in for example the embodiments to be presented hereinbelow.

In particularly attractive embodiments, the housing is configured to receive a plurality of cassettes, each with a different reagent carrier, wherein the respective reaction spaces of the plurality of cassettes extend in a row and parallel to each other, and wherein the temperature control system is configured for individual heating of the respective active indicator pad in said respective reaction spaces. In these embodiments, all the advantages of the present invention are manifested optimally. The various cassettes with various reagents may undergo their respective reaction with their milk sample at a different temperature, without affecting those adjacent. Moreover, the system as a whole can be made very compact.

In particular, the temperature control system comprises a plurality of separately controllable second optical radiation sources, in particular a second optical radiation source per cassette. This configuration offers optimal flexibility when heating the respective indicator pads. Thus, for example, samples can be applied on two or more different indicator pads shortly after each other, and each can react at the temperature that is optimal for them. These reactions may also overlap each other in time, wherein the “dedicated” second optical radiation source brings or keeps the temperature at the required level.

In some embodiments, the concentrator is adjustable, and the temperature control system is configured to concentrate the emitted optical heating radiation onto a desired active indicator pad. By adjusting the concentrator, such as by turning or tilting, this can bring the heating radiation of the second optical radiation source onto the desired indicator pad. Thus, only one second optical radiation source is required, if it has an adjusting mechanism for the concentrator. By this means it is also possible to bring and hold two or more indicator pads at a desired temperature, for example by allowing the concentrator to change between these indicator pads at a sufficiently high frequency.

The invention will now be explained in more detail based on some nonlimiting example embodiments, as well as the drawing, in which:

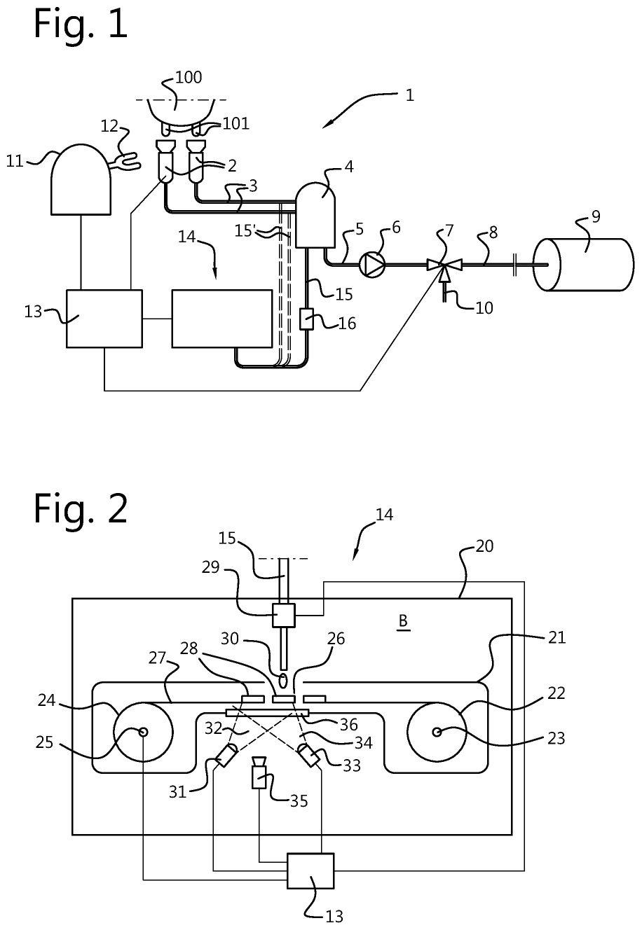

FIG. 1 shows schematically a milking system according to the invention,

FIG. 2 shows schematically a first embodiment of an analyser 14 of a milking system 1 according to the invention, and

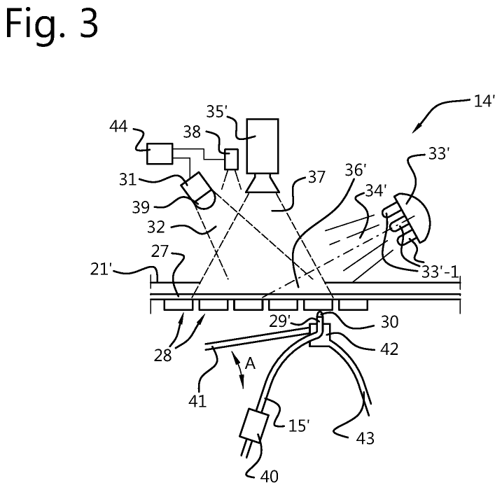

FIG. 3 shows schematically at least a part of an alternative analyser 14′.

FIG. 1 shows schematically a milking system 1 according to the invention for milking a milking animal with an udder 100 with teats 101. The milking system 1 comprises teat cups 2 and a milking robot 11 with a robot arm with a gripper 12, as well as a control system 13. Milk tubes 3 convey milk to a milk glass 4. Via a milk pipeline 5, a milk pump 6 pumps the milk via valve device 7 and a tank pipeline 8 to a milk tank 9, or to a sewer 10.

An analyser 14 receives a milk sample via a sampling device with a sample line 15 and a sample pump 16. Alternative sample lines are indicated with 15′.

The milking system 1 shown here comprises milking means in the form of a fully automatic milking device, i.e. a milking robot 11. In this, a robot arm with gripper 12 connects the teat cups 2 to the teats 101 of the milking animal. The milking robot shown has a gripper, but may also be such that a holder is provided on the robot arm for detachable placement thereon of all teat cups 2, such as in the Lely Astronaut® system. Moreover, it is possible that the milking system relates to a conventional milking system, wherein the teat cups are not connected automatically by a robot arm, but by a person. The number of teat cups 2 is generally four, such as for cows, or two, such as for goats. The milk milked with the teat cups 2 goes, under the effect of the milking vacuum, via the milk tubes 3 into the milk glass 4. In practice, the milking means comprise many other components, such as a vacuum pump, a pulsator, and so on, but these components are not important for the invention. What is relevant, of course, is the control system 13 of the milking system.

The milk that the milking system obtains from the teats 101 is thus collected during milking in the milk glass 4. After milking, the milk is pumped by the milk pump 6 via the milk pipeline 5 to a milk tank 9, or optionally to another receiver or the sewer. The choice of this is regulated by the valve gear 7, which is controlled by the control system 13, based on sampling of the milk. For sampling, a milk sample is taken from the milk in the milk glass 4 by means of the sample line 15 and the sample pump 16. For further details of this sampling device, reference is to be made to the prior art, since these details are not relevant for the present invention. It is to be noted that a sample from the milk glass 4 will always be a mixed sample. Alternatively, it is possible to take a sample from one or more milk tubes 3, by means of the associated alternative sample tube(s) 15′. This principle is also known per se.

The sample pump 16 sends the milk sample to the analyser 14. The milk sample is analysed in the latter. Based on the result of the analysis, the control system 13 may decide not to direct the milk to the milk tank 9, but for example to the sewer, or to some other milk receiving holder (not shown here). These will be explained in more detail hereunder on the basis of FIGS. 2 and 3.

FIG. 2 shows schematically a first embodiment of an analyser 14 of a milking system 1 according to the invention. Similar components are indicated in the whole drawing with the same reference numbers, optionally provided with a prime (′). The analyser comprises a housing 20 with an interior space B for receiving a cassette 21. The cassette 21 comprises a first holder in the form of a reel 22 that is rotatable about a first spindle 23, and a second holder in the form of a reel 24 that is rotatable by a motor 25. A reaction space located between the first holder and the second holder is indicated with 26. The holders 22 and 24 carry a tape 27 with indicator pads 28 thereon, onto which a metering device 29 can supply a drop 30 of milk.

A heating LED is indicated with 31, which emits heating radiation 32. An optical LED 33 emits optical radiation 34. A camera 35 takes images via a window 36.

The housing 20 is for example a windproof and waterproof housing, and has an interior space B, which advantageously is thermally insulated. In space B, for example a cassette 21 is receivable, interchangeable via a hatch (not shown). The cassette has a first holder 22 in the form of a reel with a tape 27 wound thereon, on which indicator pads 28 are applied, or alternatively may be applied from a magazine (not shown). In the latter case the tape only functions as a temporary carrier and transporter. In the former case the tape 27 itself brings in each case a new indicator pad 28 from the first holder 22 to the reaction space 26, which is between the two holders 22 and 24. For this purpose, here the second holder 24 is drivable by the motor 25, such as a stepping motor.

In the manner described above, a “fresh” or active indicator pad 28 is placed in the reaction space 26, where this can receive a drop 30. In the indicator pad 28, one or more reagents are placed, which in the presence of a certain substance in the milk drop 30 may undergo a (colour) reaction. The colour and/or intensity of the latter may depend on the concentration of the substance to be detected. A familiar example is a colour change for determining a pH. The indicator pad 28 may simply be an amount of reagent deposited on the tape 27, but often the reagent is taken up in a kind of pad of absorbent material, for example also so as to be able to distribute the milk well. Such pads may be provided separately on the tape 27, which is then rolled up on or in the first holder 22. Alternatively the indicator pads may be provided on a separate carrier (“dry stick”), which can be provided on the tape 27 from a separate magazine.

The colour or colour change may be observed by a camera 35. This is provided here on the other side than the side from where the drop 30 is provided, but this could also be the same side, wherein either the camera views at an angle, or the metering device 29 or the camera 35 is movable. To make determination of the colour (or colour change) more reliable/more reproducible, an optical light source is provided in the form of an optical LED 33, which emits optical radiation 34. This optical radiation will generally be visual radiation, such as white light, or also narrow-band radiation such as blue or red light, if the colour reaction permits. The camera 35 views in this case through a window 36 into the cassette 21, said window being transparent to at least that part of the optical radiation 34 in which the colour reaction takes place. For other wavelength ranges the window 36 does not need to be transparent, but may of course be so.

For many reactions it is favourable if these take place at a known or constant temperature. The measurements are then in principle more reliable and/or more reproducible. The reaction rate is also more controllable. Especially at low temperatures, which may quickly be reached in a milking parlor in winter, reactions are much slower, which is unfavourable if on the basis of the measurement a decision has to be taken about the milked milk, or if relatively many measurements have to be done in a short time. According to the invention, for this purpose in addition a temperature control system is provided, in the form of a heating LED 31 that emits optical heating radiation 32. This optical heating radiation 32 generally is or comprises near-infrared radiation (NIR), for reasons of efficiency and the compactness of LEDs as sources. However, other wavelengths may also be usable, in particular depending on the absorption properties of the material to be heated, in this case the at least one active indicator pad 28. These often have high absorption in the NIR region.

Temperature control may be brought about by leaving the LED 31 switched on only for a certain time, such as 5 seconds, or for a time that is dependent on the ambient temperature. The amount of energy supplied is therefore known. If in addition the absorption properties of the indicator pad 28 are known, such as from calibration measurements, the temperature to be reached may therefore also be known. The ambient temperature may, moreover, be determined with a thermometer, not shown separately here. Of course, the length of time for heating will be shorter if this temperature is higher. Control of the source 31 is performed herein by a module, not shown separately, within the control system 13, which thus forms a source control device. It is of course also possible to provide separate source control, which is connected actively to the control system 13.

FIG. 3 shows schematically at least one part of an alternative analyser 14′.

The analyser 14′ again comprises, in a housing not shown here, a cassette 21′, only a small part of which is visible, said part comprising a window in the form of an opening 36′. Moreover, a heating LED 31 is provided, now with a lens 39, which emits heating radiation 32 in a solid angle. The optical radiation source 33′ comprises some sub-LEDs 33′-1. In addition, a contactless thermometer is indicated with 38. Finally, the metering device 29′, only shown very schematically, for supplying the drop of milk 30 comprises an overflow cup 42, which is movable by means of arm 41 in the direction of the arrows A. A drip-feed pump is indicated with 40, and a discharge with 43.

This analyser may be read in light of the published application WO2020067886A1 and the further details given in that document relating to the analyser. Thus, for clarity, the housing or the first and second holders for tape, which there well might be, are not shown in FIG. 3. In this embodiment, the drop of milk 30 from the milk sample is applied from below onto the indicator pads 28, which effectively prevents milk residues getting onto the camera. The drip-feed pump 40 is to supply a drop 30 from the milk sample supplied by the sample pump, not shown here, onto the (active) indicator pad 28. For example, if the pump 40 is a peristaltic pump that is movable to and fro, so that after the drop has been supplied and has been fully absorbed by the indicator pad 28, the remaining milk can be drained off again and then led away via the discharge 43. For further details, reference should again be made to the aforementioned patent document.

In this embodiment, the camera 35′ looks through the carrier/tape 27, which in this case must therefore be transparent to the optical radiation 34′. This radiation 34′ is emitted here by part-LEDs 33′-1. For emitting white light, these will generally be different LEDs (such as RGB). This also creates the possibility of making a selection in the emitted light, for example in order to give a better colour reaction. For example, the litmus reaction from red to blue is entirely clear under (pure) red or blue light. If the analyser 14′ is only intended for a single kind of colour reaction, it is also possible to choose a narrow-band source 33′, such as with only a single colour sub-LED 33′-1.

The temperature control device again comprises an LED 31 as radiation source for the heating radiation 32. This is directed by means of a lens 32 in a relatively narrow solid angle, narrow enough in principle to illuminate and thus heat exclusively the active indicator pads 28 that are visible through the window 36′. Note that this field to be illuminated may thus also be elongated, depending on the shape of the indicator pads 28, so that the lens 39 may also be for example a cylindrical lens or mirror, or something similar. It should be emphasised here that the relative dimensions of the sources 31 and 33′ do not reflect reality. Since in practice the heating source 31 will have a higher power than the optical radiation source 33′, the former will usually also be bigger.

The temperature control device further comprises a thermometer, here a contactless thermometer 38, such as an infrared radiation thermometer. This is able to measure the temperature of (the surface of) the tape 27. Since the tape 27 is very thin, this is a good approximation of the temperature of the indicator pad(es) 28 located on the other side. Thus, the thermometer 38 in fact measures the temperature at which the colour reaction of milk with the one or more reagents takes place in the indicator pad 28. This temperature is preferably always as identical as possible, so as to obtain a measurement that is as reproducible and reliable as possible. Particularly in view of the circumstance that the analyser 14′ will usually be placed in an animal house environment, which is often exposed to weather effects, the ambient temperature could be very variable, so that good temperature control prevents the reaction taking place very variably. This could be compensated with a correction based on calibration measurements, but a more accurate measurement at constant temperature is preferred.

The temperature control device is thus configured here to control the LED 31 on the basis of the temperature measured by the thermometer 38. This happens by means of the source control device 44. The latter may also be a module within the control system 13 (not shown here). It is important to note that the source control device 44, and thus at least the control system 13, can ensure that LED 31 does not emit heating radiation simultaneously with the source 33′, at least not simultaneously with detection by the camera 35′. Also on account of the very high speed of reaction of LEDs, this is easy to achieve in practice.

The reaction in the indicator pad 28 takes a certain time, sometimes up to a good 15 minutes. In this time, it may certainly happen that a subsequent sample should already be taken, and the indicator pad moved on a bit, to the left in the drawing. For as long as the reaction should last, the pad 28 should also be kept at temperature. Therefore the window 36′ should be large enough to keep a plurality of pads 28 visible to the heating LED 31 and the camera 35′. These visible indicator pads may be designated as “active indicator pads”, in contrast to the used pads and naturally the pads that have not yet been used.

1. A milking system, comprising:
a milking device with milking means and a milking control device, said milking device being configured to extract milk from a milking animal,
a sampling device that is configured to take a sample of milk extracted by the milking device, and
an analyser that is configured to analyse the sample,
the analyser comprising:
a housing for receiving at least one first holder with a reagent carrier with indicator pads applied thereon, as well as for receiving a second holder for collecting used reagent carrier, wherein the indicator pads comprise a reagent that in the presence of at least one substance in the milk of the sample gives a detectable reaction,
a reaction space provided within the housing between the first holder and the second holder, for containing a part of the reagent carrier with at least one active indicator pad,
a metering system for supplying the sample taken to one of the indicator pads,
a first optical radiation source for emitting optical imaging radiation onto said active indicator pad,
an optical sensor device configured to detect optical response radiation that comes from the active indicator pad in response to the emitted optical imaging radiation, and to analyse the detected optical response radiation for supplying an indication of a presence or concentration of said at least one substance in the sample, and
a temperature control system that is configured to bring or maintain the active indicator pad at a desired temperature, the temperature control system comprising comprises:
a second optical radiation source that is configured to emit optical heating radiation at a first solid angle,
a concentrator for concentrating emitted optical heating radiation at a smaller second solid angle and onto the active indicator pad, and
a source control device that is configured to control the second optical radiation source.

2. The milking system as claimed in claim 1, wherein the first optical radiation source and the second optical radiation source are provided within the housing, wherein each cassette comprises a window that, when using the milking system, allows the optical imaging radiation and/or the optical heating radiation to pass through.

3. The milking system as claimed in claim 1, wherein the temperature control system is configured to have at any time at most one of the first optical radiation source and the second optical radiation source in operation.

4. The milking system as claimed in claim 1, wherein the temperature control system further comprises a thermometer configured for determining a temperature of the active indicator pad, and that is configured to control the second optical radiation source on the basis of the temperature determined.

5. The milking system as claimed in claim 1, wherein the temperature control system is configured to emit the optical heating radiation for a predetermined length of time.

6. The milking system as claimed in claim 1, further comprising a second thermometer for determining an ambient temperature, and wherein the temperature control system is configured to control the second optical radiation source on the basis of the ambient temperature determined.

7. The milking system as claimed in claim 1, wherein the second optical radiation source comprises an LED.

8. The milking system as claimed in claim 1, wherein the concentrator is a concave mirror or a lens.

9. The milking system as claimed in claim 1, wherein said second solid angle has a widest apex angle of at most 15.

10. The milking system as claimed in claim 1, wherein the housing is configured to receive a plurality of cassettes, each with a different reagent carrier, wherein respective reaction spaces of the plurality of cassettes extend in a row and parallel to each other, and wherein the temperature control system is configured for individual heating of a respective active indicator pad in said respective reaction spaces.

11. The milking system as claimed in claim 10, wherein the temperature control system comprises a plurality of separately controllable second optical radiation sources.

12. The milking system as claimed in claim 10, wherein the concentrator is adjustable, and wherein the temperature control system is configured for concentrating the emitted optical heating radiation onto a desired active indicator pad.

13. The milking system as claimed in claim 1, wherein the first holder and the second holder are assembled in an interchangeable cassette.

14. The milking system as claimed in claim 2, wherein the first optical radiation source and the second optical radiation source are provided outside each cassette.

15. The milking system as claimed in claim 1, wherein the temperature control system is configured to emit the optical heating radiation for a predetermined length of time depending on the reagent.

16. The milking system as claimed in claim 7, wherein the LED is a near-infrared LED.

17. The milking system as claimed in claim 1, wherein said second solid angle has a widest apex angle of at most 10°.

18. The milking system as claimed in claim 10, wherein the temperature control system comprises a separately controllable second optical radiation sources per cassette.
